(19) United States
(12) Patent Application Publication
(10) Pub. No.: US 2009/0223515 A1
(43) Pub. Date: Sep. 10, 2009

(54) NON-EJECTABLE LIQUID CARTRIDGE AND LIQUID EJECTION APPARATUS

(75) Inventor: Shinji Watanabe, Oita-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/398,005

(22) Filed: Mar. 4, 2009

(30) Foreign Application Priority Data
Mar. 10, 2008 (JP) 2008-059478

(51) Int. Cl. A61M 15/00

(52) U.S. Cl. 128/203.15

(57) ABSTRACT

An inhaler includes an ejection head having a liquid ejection nozzle and a liquid solution passage communicating with the liquid ejection nozzle. A liquid solution cartridge and a non-ejectable liquid cartridge are attached to the head interchangeably for supplying a liquid solution and a non-ejectable liquid such as a cleaning liquid, respectively. The non-ejectable liquid cartridge includes a non-ejectable liquid supply section and a non-ejectable liquid recovery section adjacently to each other. When the non-ejectable liquid cartridge is attached to the ejection head, the non-ejectable liquid supply section communicates with the liquid solution passage via an openable sealing member and then the non-ejectable liquid recovery section communicates with the liquid solution passage via a second openable sealing member. The second openable sealing member is provided between the non-ejectable liquid supply section and the non-ejectable liquid recovery section.

### BACKGROUND OF THE INVENTION

1. Field of the Invention

The present invention relates to a non-ejectable liquid cartridge of liquid ejection apparatus, which is constructed so that a user may carry and possess it, and which is used for an inhaler etc. which ejects a medicine as minute liquid droplets and makes the medicine inhaled, and a liquid ejection apparatus including the cartridge.

2. Description of the Related Art

An inhaler is developed, which makes minute liquid droplets of a liquid medicine (liquid solution) ejected in an airflow passage, through which air inhaled through a mouthpiece flows, using an ejection principle of an inkjet system to make a user inhale the minute liquid droplets (refer to Japanese Patent Application Laid-Open No. 2004-290593). Such an inhaler has an advantage that it can spray predetermined amount of liquid medicine accurately in an equalized particle diameter.

As fundamental construction of such a liquid ejection apparatus, there are an ejection head in which an ejection energy generating element, such as a heat-generating element, is arranged, and a liquid solution cartridge including a liquid medicine tank which contains a liquid medicine supplied to the ejection head.

Since these apparatuses are those of supplying a liquid solution to lungs, it is required that an ejection nozzle, a liquid solution passage, etc. of an ejection head are clean similarly to a syringe. In particular, when repeatedly using an ejection head over a long period of time, there is a possibility that a problem in a sanitary aspect may occur.

Heretofore, when an ejection head is cleaned, since a valve mechanism which switches tanks in which a liquid solution and cleaning liquid are contained, and a pump mechanism which performs pressure or attraction for exchanging the liquid solution and a cleaning agent by turns become necessary, an apparatus itself is upsized as disclosed in Japanese Patent Application Laid-Open No. 10-024614. In addition, as for what was disclosed in Japanese Patent Application Laid-Open No. 2004-268351, it was necessary to prepare complicated structure for clearing in an ejection head side.

### SUMMARY OF THE INVENTION

The present invention aims at providing a non-ejectable liquid cartridge, which is attached to an ejection head instead of a liquid solution cartridge, can supply a cleaning agent, and can recover the cleaning agent after cleaning, and a liquid ejection apparatus including the cartridge.

The non-ejectable liquid cartridge of the present invention is a non-ejectable liquid cartridge adapted for being attached to an ejection head of a liquid ejection apparatus, comprising: a non-ejectable liquid supply section which is filled with a non-ejectable liquid; a non-ejectable liquid recovery section for recovering the non-ejectable liquid; a first openable sealing member through which the non-ejectable liquid supply section can communicate with a liquid solution passage of the ejection head in order to supply the non-ejectable liquid to the ejection head from the non-ejectable liquid supply section; and a second openable sealing member through which the non-ejectable liquid recovery section can communicate with the liquid solution passage of the ejection head in order to recover the non-ejectable liquid into the non-ejectable liquid recovery section from the ejection head, wherein the non-ejectable liquid recovery section is arrange adjacent to the non-ejectable liquid supply section and the second openable sealing member is provided between the non-ejectable liquid supply section and the non-ejectable liquid recovery section.

In construction of using an ejection head multiple times and using only a liquid solution cartridge wastefully, after removing the liquid solution cartridge after inhalation, a disposable non-ejectable liquid cartridge is attached. First, the first openable sealing member (thin membrane) for the non-ejectable liquid supply section in which a cleaning liquid (non-ejectable liquid) is filled is made to be penetrated by a penetrating member which constructs the liquid solution passage of the ejection head, and a cleaning liquid is made to be filled in the ejection head. Next, the cleaning liquid is made to flow backwards from the ejection head, and to be absorbed by a liquid holding means via the second openable sealing member (thin membrane) for the non-ejectable liquid recovery section being provided with the liquid holding means in negative pressure, with a liquid absorber, etc., and being made to be penetrated by the above-mentioned penetrating member.

The ejection nozzle and liquid solution passage of the ejection head can be cleaned simply and can be repeatedly used sanitarily by the non-ejectable liquid cartridge being attached by the same operation as the case that the liquid solution cartridge is attached at the time of normal use. In addition, deposition and staining can be prevented even when long-term storage is performed, by the cleaning liquid being made to be absorbed by the liquid holding means of the non-ejectable liquid recovery section, and being removed from an inside of the ejection head.

By one action of operation of attaching the non-ejectable liquid cartridge to the ejection head, a series of operations of cleaning the ejection head and recovering the non-ejectable liquid are achievable.

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIGS. 1A and 1B are diagrams illustrating a liquid solution cartridge and a non-ejectable liquid cartridge of a liquid ejection apparatus according to a first embodiment.

FIGS. 2A, 2B, 2C, and 2D are process charts illustrating process steps of attaching the liquid solution cartridge in FIG. 1A to an ejection head.

FIGS. 3A, 3B, 3C, and 3D are process charts illustrating process steps of attaching the non-ejectable liquid cartridge in FIG. 1B to the ejection head.

FIG. 4 is a diagram illustrating a principal section in a second embodiment.

### DESCRIPTION OF THE EMBODIMENTS

Preferred embodiments of the present invention will now be described in detail in accordance with the accompanying drawings.

### Embodiment 1

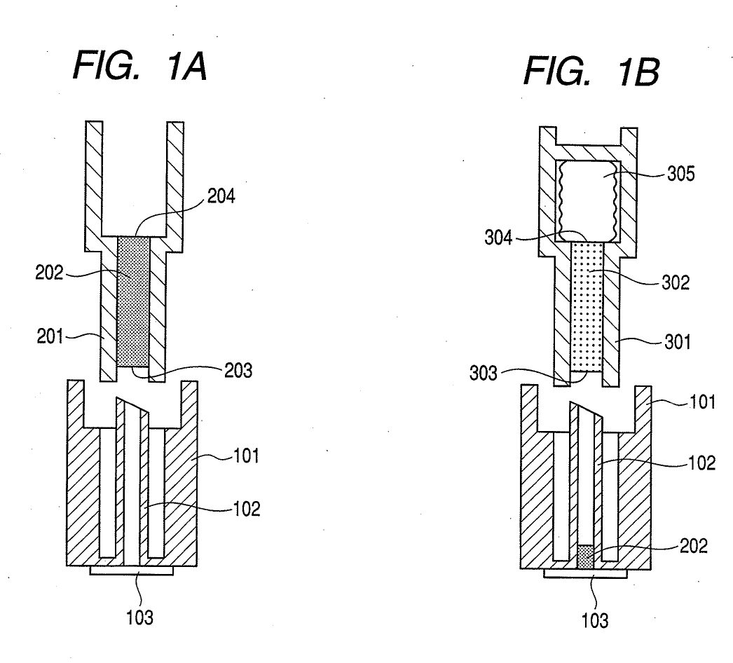

FIGS. 1A and 1B, FIGS. 2A to 2D, and FIGS. 3A to 3D describe the first embodiment. An ejection head 101 has a liquid solution passage formed inside a penetrating member 102 constructed in one piece with a main body of an ejection head, and a liquid solution ejecting section 103 including an ejection nozzle and an ejection energy generating element. A disposable liquid solution cartridge 201 contains a liquid solution 202, and has first and second thin membranes 203 and 204 for making the liquid solution 202 filled into the liquid solution cartridge 201.

A non-ejectable liquid cartridge 301 which can be attached to the ejection head 101 by which the liquid solution cartridge 201 is replaced has a non-ejectable liquid supply section filled with a cleaning liquid 302, or a cleaning/sterilizing liquid which is a non-ejectable liquid. In addition, the non-ejectable liquid cartridge 301 includes thin membranes 303 and 304 which are first and second openable sealing members for holding the cleaning liquid 302 in the above-mentioned non-ejectable liquid supply section, and a non-ejectable liquid recovery section 305 arranged adjacent to the above-mentioned non-ejectable liquid supply section via the thin membrane 304 which is the second openable sealing member. Thus, the non-ejectable liquid supply section is a space enclosed with the thin membrane 303 and thin membrane 304 inside the non-ejectable liquid cartridge 301. A non-ejectable liquid recovery section 305 is a space to which negative pressure is given, and has a liquid holding means, such as a liquid absorber.

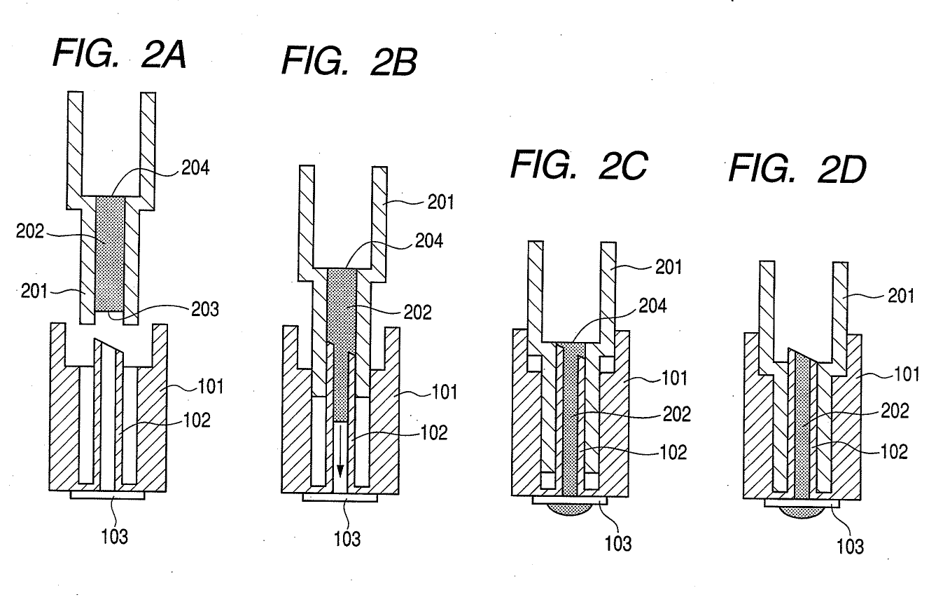

FIGS. 2A to 2D illustrate an attachment method of the liquid solution cartridge 201 at the time of normal use of the liquid ejection apparatus in an inhaler for inhaling a liquid solution which is a medicine. The ejection head 101 and liquid solution cartridge 201 which are separated as illustrated in FIG. 2A are coupled. By this operation, first, as illustrated in FIG. 2B, the first thin membrane 203 of the liquid solution cartridge 201 is broken by the penetrating member 102 which constructs the liquid solution passage of the ejection head 101, and the liquid solution 202 goes into an inside (liquid solution passage) of the penetrating member 102. At this time, when an inner diameter of the liquid solution cartridge 201 is not larger than an outer diameter of the penetrating member 102, the liquid solution 202 does not leak to outside, but goes into an inside of the penetrating member 102 altogether.

When one of the penetrating member 102 and liquid solution cartridge 201 is a hard rigid body and the other is an elastic body etc., the elastic one deforms, both stick fast, and liquid leakage is prevented. Construction that the penetrating member 102 is hard and the liquid solution cartridge 201 is elastic is desirable. In such a case, the inner diameter of the liquid solution cartridge 201 and the outer diameter of the penetrating member 102 do not need to be equal.

As illustrated in FIG. 2C, by further pushing the liquid solution cartridge 201, the liquid solution 202 permeates the inside of the head to be filled in the minute passage and ejection nozzle of the liquid solution ejecting section 103. A processing unit (not illustrated) of the liquid solution 202 overflowing from the ejection head 101 which uses a wiper, a liquid absorber, etc. shall be prepared separately. The penetrating member 102 breaks through the second thin membrane 204 of the liquid solution cartridge 201 eventually, and the liquid solution 202 communicates with atmospheric air. Thus, the penetrating member 102 has sufficient length to break through the second thin membrane 204.

The first and second thin membranes 203 and 204 can be penetrated simply by the penetrating member 102. As materials used for the thin membranes, polymeric materials, such as polyethylene and cycloolefin polymers, can be exemplified. The liquid solution 202 is given ejection energy by the ejection energy generating element in this state, and becomes fine liquid droplets to be inhaled by a user. Since the second thin membrane is broken and the liquid solution 202 is communicating with atmospheric air, the same amount of the liquid solution as that being ejected and consumed is supplied from the back one by one. After finishing inhaling the liquid solution like this, the liquid solution cartridge 201 is removed and discarded.

Here, an electrothermal transducer which gives heat energy to a medicine which is a liquid solution typically is mentioned as the ejection energy generating element. This has a function of ejecting a medicine with using a so-called principle of a thermal inkjet system. In addition, it is sufficient to use a so-called piezo jet system that gives mechanical energy to a medicine by a piezoelectric element or the like which is an electromechanical transducer to perform ejection. In addition, it is no matter to apply an ejection principle of a conventional metered-dose inhaler (MDI), nebulizer, dry powder inhaler (DPI), or the like.

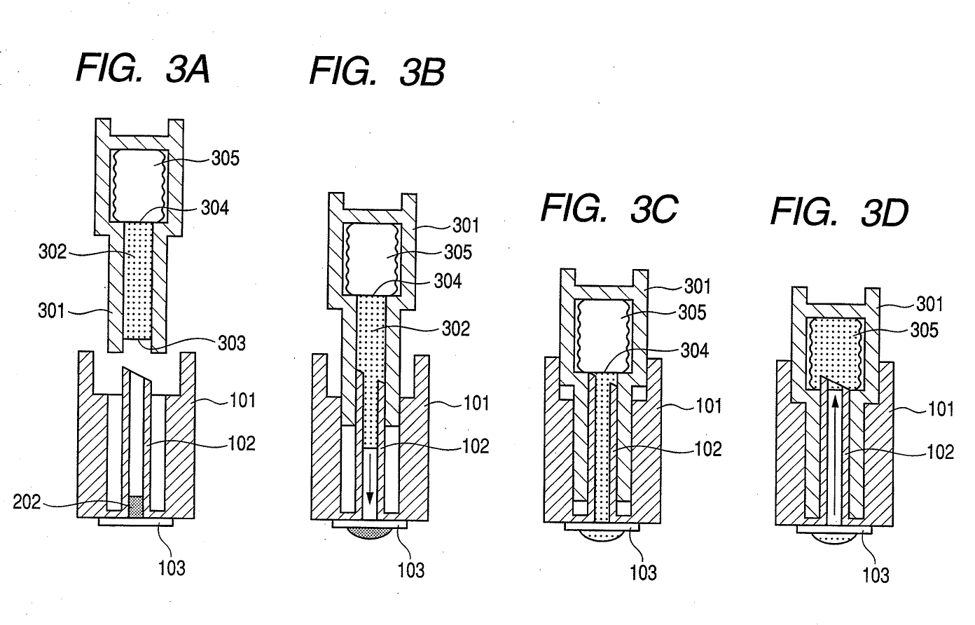

FIGS. 3A to 3D illustrate an attachment method of the non-ejectable liquid cartridge 301 at the time of clearing by performing supply and recovery of a cleaning liquid after use of an inhaler. Similarly to the case of using the liquid solution cartridge 201, the ejection head 101 and the non-ejectable liquid cartridge 301 which are separated as illustrated in FIG. 3A are coupled. First, as illustrated in FIG. 3B, the thin membrane 303 which is the first openable sealing member of the non-ejectable liquid cartridge 301 is broken through by the penetrating member 102 of the ejection head 101, the non-ejectable liquid supply section communicates with the liquid solution passage of the penetrating member 102, and the cleaning liquid 302 goes into the inside of the ejection head 101. In the case of a protein pharmaceutical preparation etc., which have a possibility of decay of the liquid solution 202, instead of the cleaning liquid 302, it is also good to use a cleaning/sterilizing liquid in which an antiseptic like alcohol, cresol, or the like is contained.

Various surface active agents can be used as the cleaning liquid. In addition, benzalkonium chloride is mentioned as the surface active agent which can be used also as the antiseptic solution. However, it is good to use as these cleaning liquids what is preferable according to a type of a medicine to be ejected, and there is no limitation.

As illustrated in FIG. 3C, the cleaning liquid 302 permeates the inside of the head by the non-ejectable liquid cartridge 301 being further pushed, is filled in the minute passage and ejection nozzle of the liquid solution ejecting section 103, and dilutes and removes the liquid solution 202 which remains inside the ejection head 101.

Eventually, as illustrated in FIG. 3D, the penetrating member 102 breaks through the thin membrane 304 which is the second openable sealing member which intervenes between the non-ejectable liquid supply section and the non-ejectable liquid recovery section 305 of the non-ejectable liquid cartridge 301, and communicates with the non-ejectable liquid recovery section 305. The non-ejectable liquid recovery section 305 includes, for example, a liquid absorber to which negative pressure is given. In addition, the non-ejectable liquid recovery section 305 is separated by the first thin membrane 303 and the second thin membrane 304 from atmospheric air, and the given negative pressure is maintained at the time of intactness. When the negative pressure given to the non-ejectable liquid recovery section 305 is lower than an atmospheric pressure enough, the cleaning liquid 302 flows backwards the penetrating member 102, and is recovered and held promptly in the non-ejectable liquid recovery section 305. When being held until next time use while being integrated, this non-ejectable liquid cartridge 301 can be used as a cap which prevents invasion of contamination from the penetrating member 102.

In order for the penetrating member 102 to penetrate both the thin membrane 304 and the thin membrane 305, it is good just to make a length of the penetrating member 102 longer than a distance between the thin membrane 304 and the thin membrane 305.

Here, it is preferable that a volume of the space between the first and second thin membranes which is the non-ejectable liquid supply sections is smaller than a volume of the non-ejectable liquid recovery section 305. Such construction enables to recover all the non-ejectable liquid. When accommodating the liquid absorber in the non-ejectable liquid recovery section 305, it is regarded that a volume by which the liquid absorber can absorb a liquid is the above-mentioned volume of the non-ejectable liquid recovery section 305.

In order to recover the non-ejectable liquid when the thin membrane 304 is penetrated, negative pressure is given to an inside of the non-ejectable liquid recovery section 305. Although it is an example of providing a liquid absorber in an inside, it is not limited to it, but, for example, internal pressure may be reduced at the time of manufacturing a cartridge. The negative pressure means pressure smaller than that of an outside of the non-ejectable liquid recovery section 305.

Cleaning of the liquid solution ejecting section of the ejection head 101 and the penetrating member 102 can be performed simply in the same procedure as the case that the liquid solution is inhaled. Since mechanisms such as a valve and a pump are not needed, not only the apparatus is not upsized, but also it is not necessary to give special structure for clearing to the ejection head 101, and to prepare a dedicated cleaning station.

### Embodiment 2

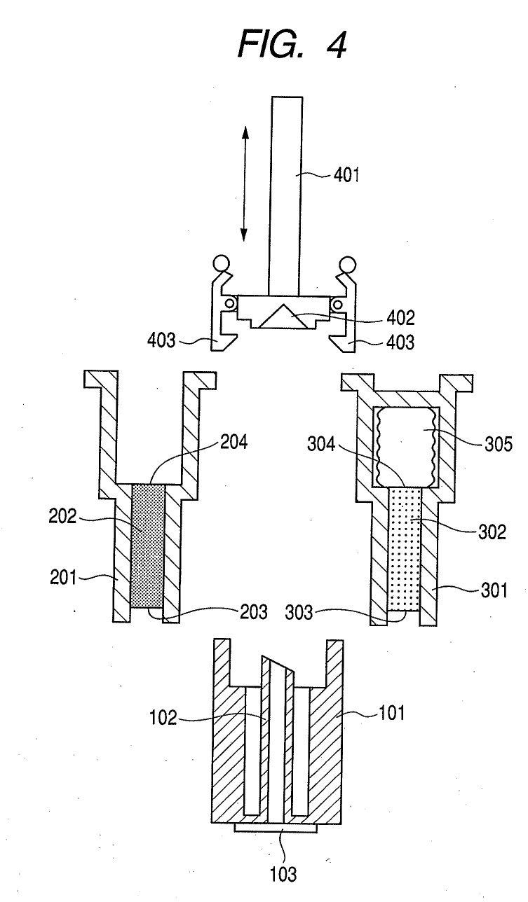

FIG. 4 is a diagram illustrating a second embodiment. Inside a liquid ejection apparatus which has the same ejection head 101 as that of the first embodiment, a mounter including a transfer shaft 401, a sensor 402, and a hook 403 is provided. Although the sensor 402 is a reflection type, it may be another type, such as a push button type.

According to the procedure illustrated in FIGS. 2A to 2D and FIGS. 3A to 3D, the liquid solution cartridge 201 and the non-ejectable liquid cartridge 301 are coupled with the ejection head 101, respectively, and are used. For the purpose of use, when using the liquid solution cartridge 201, since a tip of the penetrating member 102 communicates with atmospheric air, a rear edge of the liquid solution cartridge 201 is opened. On the other hand, a rear edge of the non-ejectable liquid cartridge 301 contains the non-ejectable liquid recovery section 305, and hence, it needs to be sealed. The transfer shaft 401 provided inside the liquid ejection apparatus is connected to a rear edge of each cartridge, and can automatically couple the cartridge with the ejection head 101. The hook 403 is provided at a tip of the transfer shaft 401, and the ejection head 101 and the each cartridge are separable. Since being provided also at the tip of the transfer shaft 401, the sensor 402 enables to discriminate a type of a connected cartridge because of a rear edge form of the each cartridge. Even if the liquid solution cartridge 201 or non-ejectable liquid cartridge 301 is loaded inside the liquid ejection apparatus at random, its type can be discriminated by the sensor 402. Hence, the liquid ejection apparatus can select a cartridge according to an operating condition automatically, and can perform a coupling/separating/holding operation automatically.

Since coupling/separation of the ejection head 101 and each cartridge can be performed in a simple operation, it can be performed manually, but the automatic operation by the transfer shaft 401 also enables a weak child or an aged person to use the apparatus. In addition, since coupling can be performed by a fixed force, penetration of the liquid solution 202 and cleaning liquid 302 is performed at a fixed speed. Alternatively, by operating a processing unit (not illustrated) of the liquid solution or cleaning liquid overflowing from the ejection head 101 with interlocking with pushing quantity of the transfer shaft 401, stain inside the liquid ejection apparatus can be suppressed to the minimum.

The present invention is not limited to the above embodiments and various changes and modifications can be made within the spirit and scope of the present invention. Therefore to apprise the public of the scope of the present invention, the following claims are made.

This application claims the benefit of Japanese Patent Application No. 2008-059478, filed Mar. 10, 2008, which is hereby incorporated by reference in its entirety.

What is claimed is:

1. A non-ejectable liquid cartridge adapted for being attached to an ejection head of a liquid ejection apparatus, comprising:
a non-ejectable liquid supply section which is filled with a non-ejectable liquid;
a non-ejectable liquid recovery section for recovering the non-ejectable liquid;
a first openable sealing member through which the non-ejectable liquid supply section can communicate with a liquid solution passage of the ejection head in order to supply the non-ejectable liquid to the ejection head from the non-ejectable liquid supply section; and
a second openable sealing member through which the non-ejectable liquid recovery section can communicate with the liquid solution passage of the ejection head in order to recover the non-ejectable liquid into the non-ejectable liquid recovery section from the ejection head,
wherein the non-ejectable liquid recovery section is arranged adjacent to the non-ejectable liquid supply section and the second openable sealing member is provided between the non-ejectable liquid supply section and the non-ejectable liquid recovery section.

2. The non-ejectable liquid cartridge according to claim 1, wherein the non-ejectable liquid is a cleaning liquid for cleaning an ejection nozzle and the liquid solution passage of the ejection head.

3. The non-ejectable liquid cartridge according to claim 1, wherein the non-ejectable liquid is a cleaning/sterilizing liquid for cleaning and disinfecting an ejection nozzle and the liquid solution passage of the ejection head.

4. The non-ejectable liquid cartridge according to claim 1, wherein the non-ejectable liquid recovery section is given negative pressure for recovering the non-ejectable liquid.

5. The non-ejectable liquid cartridge according to claim 4, wherein the non-ejectable liquid recovery section has a liquid absorber for absorbing the non-ejectable liquid.

6. The non-ejectable liquid cartridge according to claim 1, wherein the first and second openable sealing members each have a thin membrane adapted for being penetrated by a penetrating member which forms the liquid solution passage of the ejection head.

7. A liquid ejection apparatus, comprising:
an ejection head having an ejection nozzle and a liquid solution passage for guiding a liquid solution to the ejection nozzle;
a liquid solution cartridge adapted for being attached to the ejection head for supplying a liquid solution to the ejection head; and
a non-ejectable liquid cartridge adapted for being attached to the ejection head for supplying and recovering a non-ejectable liquid to and from the ejection head,
wherein the non-ejectable liquid cartridge comprises:
a non-ejectable liquid supply section which is filled with a non-ejectable liquid;
a non-ejectable liquid recovery section for recovering the non-ejectable liquid;
a first openable sealing member through which the non-ejectable liquid supply section can communicate with the liquid solution passage of the ejection head in order to supply the non-ejectable liquid to the ejection head from the non-ejectable liquid supply section; and
a second openable sealing member through which the non-ejectable liquid recovery section can communicate with the liquid solution passage of the ejection head in order to recover the non-ejectable liquid into the non-ejectable liquid recovery section from the ejection head, and
wherein the non-ejectable liquid recovery section is arranged adjacent to the non-ejectable liquid supply section and the second openable sealing member is provided between the non-ejectable liquid supply section and the non-ejectable liquid recovery section.

8. The liquid ejection apparatus according to claim 7, further comprising a mounter for attaching the non-ejectable liquid cartridge and the liquid solution cartridge to the ejection head by turns.
